(19) United States
(12) Patent Application Publication
(10) Pub. No.: US 2022/0000763 A1
(43) Pub. Date: Jan. 6, 2022

(54) SUBLINGUAL AND BUCCAL DOSAGE FORMS OF CANNABINOID EXTRACTS AND METHOD OF USE THEREOF

(71) Applicant: Columbia Care LLC, New York, NY (US)

(72) Inventor: Aaron Michael Dely, Lighthouse Point, FL (US)

(21) Appl. No.: 17/291,387

(22) PCT Filed: Nov. 7, 2019
(86) PCT No.: PCT/US2019/060313

§ 371 Date: May 5, 2021

Related U.S. Application Data
(60) Provisional application No. 62/757,069, filed on Nov. 7, 2018.

(51) Int. Cl. A61K 9/00; A61K 31/352; A61K 31/05; A61K 9/08; A61K 47/14; A61K 47/10; A61K 47/26; A61K 47/24

(52) U.S. Cl. CPC A61K 9/006; A61K 31/352; A61K 31/05; A61K 47/24; A61K 47/14; A61K 47/10; A61K 47/26; A61K 9/08

(57) ABSTRACT

The invention relates to compositions for sublingual and/or buccal administration comprising at least one cannabinoid or cannabinoid extract, triglyceride, alcohol, and at least one surfactant.

### CROSS-REFERENCE TO RELATED APPLICATIONS

This application claims the benefit of U.S. Provisional Application No. 62/757,069, filed Nov. 7, 2018, the entire contents of which are incorporated by reference herein.

### FIELD OF THE INVENTION

The invention relates to compositions for sublingual and/or buccal administration comprising at least one cannabinoid or cannabinoid extract, triglyceride, alcohol, and at least one surfactant.

### BACKGROUND OF THE INVENTION

The medicinal and psychoactive properties of the cannabis plant have been known for centuries. While it has been illegal in many countries, there is a growing populous to lobby for legalization of its use, especially for medicinal purposes.

Cannabis is believed to provide benefits in the treatment of multiple disorders with safer and fewer serious side effects than most prescription drugs currently used as antiemetics, muscle relaxants, hypnotics and analgesics. A disadvantage in treating patients with cannabis is the psychoactive effect, especially in “naive” cannabis users. Furthermore, there have been reports of unpleasant reactions to cannabis, such as anxiety, panic or hallucinations. It is believed that the undesirable side effects are most commonly associated with higher doses of cannabis and are related to the difficulty in controlling the dosage when the drug is smoked or eaten in cannabis-enriched confectionaries.

Cannabis has also been used to treat the symptoms in patients suffering from serious medical conditions. For example, cannabis has been used to alleviate symptoms associated with cancer, anorexia, AIDS, chronic pain, muscle spasticity, glaucoma, arthritis, migraine and many other illnesses. Cannabis is recognized as having anti-emetic properties and has been successfully used to treat nausea and vomiting in cancer patients undergoing chemotherapy. Cannabis has also been reported in treating the weight loss syndrome of AIDS and for the treatment of glaucoma by reducing intraocular pressure. Cannabis is also known for it muscle relaxing and anti-convulsant effects.

The most prevalent mode of administration of medical cannabis is by smoking. This mode of administration can have adverse effects on the lungs. Cannabis smoke carries more tar and other particulate matter than tobacco and may be a cause of lung diseases including lung cancer. Furthermore, many patients find the act of smoking unappealing, as well as generally unhealthy.

Accordingly, there is significant interest in developing other means to administer cannabis to patients.

Absorption of drugs from the oral cavity into the mucosal tissues is typically a fast event. Dissolved drugs partition into the mucosal membranes and within minutes will reach equilibrium with drug in solution in the oral cavity. However, this does not always equate to rapid drug appearance in the systemic circulation. This has been attributed to slow partitioning out of the mucosal tissues and into the systemic circulation. The principle barrier is the stratum corneum and one of the strategies that have been found to promote cutaneous drug penetration is through the use of absorption enhancers. A variety of solvents have been identified as “absorption enhancers”. The extraction of lipids with the formation of “pores” within the stratum corneum, thus increasing cutaneous hydration and solubility of the solute are general action mechanisms which have been suggested to explain the enhancing action of these substances. The transport of non-polar materials, such as the cannabinoids, may be increased through the addition of small quantities of fatty acids or alcohols within the products formulation. The addition of small quantities of alcohol produces a significant increase in the flow of drug through the skin, being that the longer the lateral carbon chain, this being up to a length of 14 carbon atoms, the greater is the enhancing effect produced.

Current marketed tinctures that contain decarboxylated cannabis plant material exist as oil-based tinctures or alcohol-based tinctures. Emulsions are mixtures of two or more liquids in which one is present as droplets, of microscopic or ultramicroscopic size, distributed throughout the other. Emulsions are formed from the component liquids either spontaneously or, more often, by mechanical means, such as agitation, provided that the liquids that are mixed have no mutual solubility. Emulsions are stabilized by agents that form films at the surface of the droplets or that impart to them a mechanical stability. Unstable emulsions eventually separate into two liquid layers.

In oil-ethanol emulsions, ethanol is the polar phase and oil is the nonpolar phase. The primary advantage of oil-ethanol emulsions over conventional water-oil emulsions is that they enable the incorporation of water-and oil-insoluble or poorly soluble functional compounds and/or drugs into emulsions. A non-ionic surfactant was used to stabilize the oil-ethanol emulsion.

In the past, oil and alcohol have been combined in the presence of various emulsifiers to produce an emulsion, but the results have been unsatisfactory. The oil phase separated out of the mixture after relatively short periods of time indicating an unstable emulsion.

There remains a need in the art, therefore, for an improved sublingual and buccal dosage forms for cannabinoid extracts.

### SUMMARY OF THE INVENTION

In one aspect, the invention provides a composition comprising: at least one cannabinoid or cannabinoid extract; triglyceride; alcohol; and at least one surfactant. In some embodiments, the composition is a pharmaceutical composition or formulation, such as a composition formulated in a dosage form or a unit dose.

In one aspect, the invention provides a method for treating a disease comprising administering a daily therapeutically effective amount of the pharmaceutical composition or the dosage form or the unit dose.

In another aspect, the invention provides a method of sublingually or buccally administering a therapeutic agent to a patient comprising the steps of: preparing the pharmaceutical composition and administering the pharmaceutical composition to the underside of the tongue of the patient. In some embodiments, the pharmaceutical composition is a liquid.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

The present invention will be more fully understood with reference to the following detailed description which is accompanied by drawings.

FIGS. 1-9 contain graphs displaying the cannabinoid profile of pharmaceutical formulations in tinctures containing two cannabinoids in a ratio of 20:1 THC to CBD by weight.

FIGS. 10-16 contain graphs displaying the cannabinoid profile of pharmaceutical formulations in tinctures containing two cannabinoids in a ratio of 1:1 THC to CBD by weight.

FIGS. 17-23 contain graphs displaying the cannabinoid profile of pharmaceutical formulations in tinctures containing two cannabinoids in a ratio of 1:20 THC to CBD by weight.

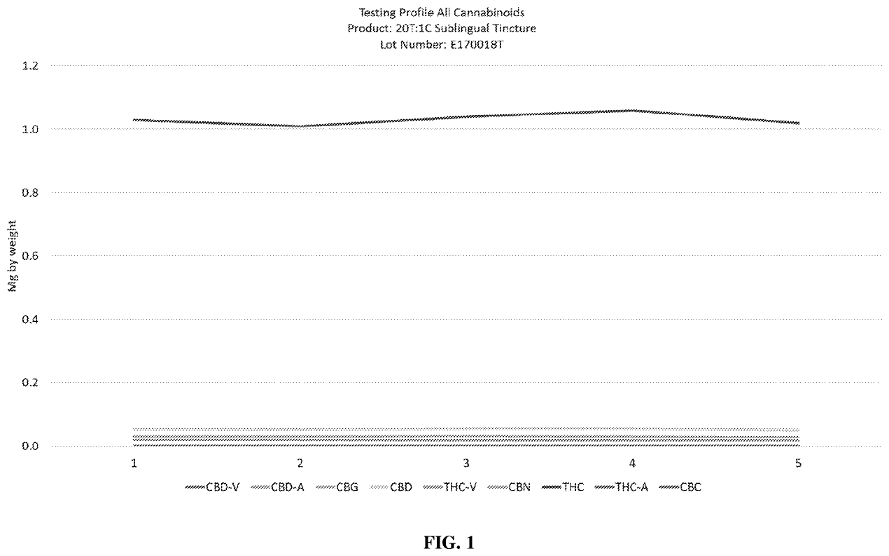

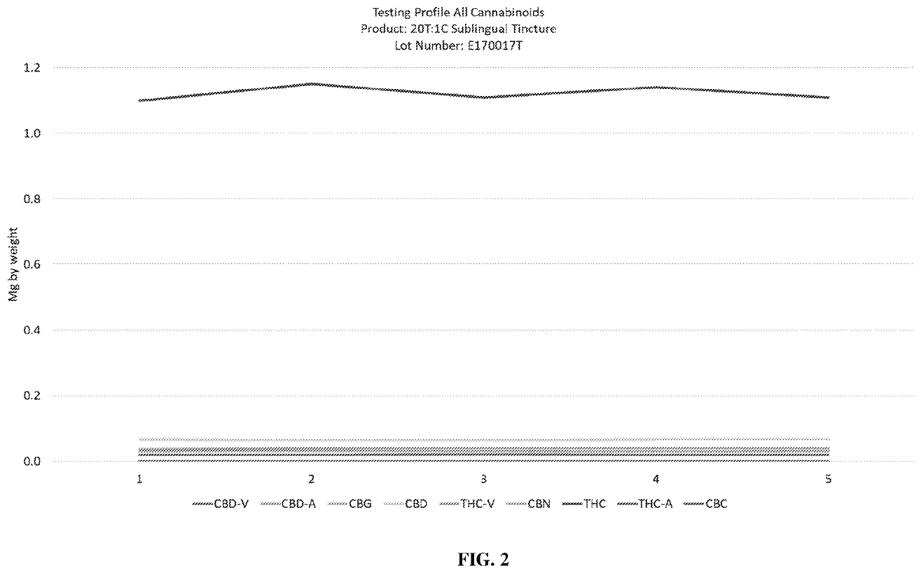

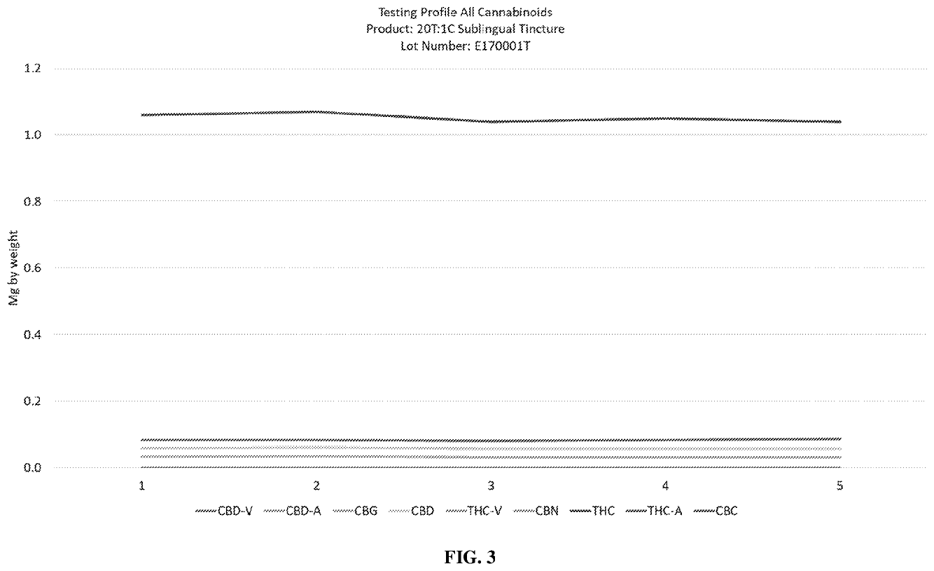

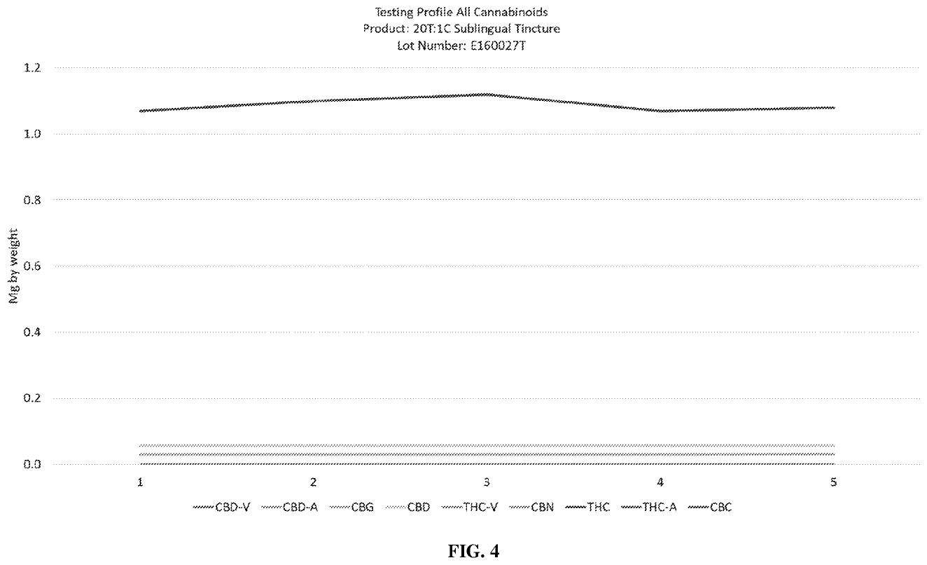

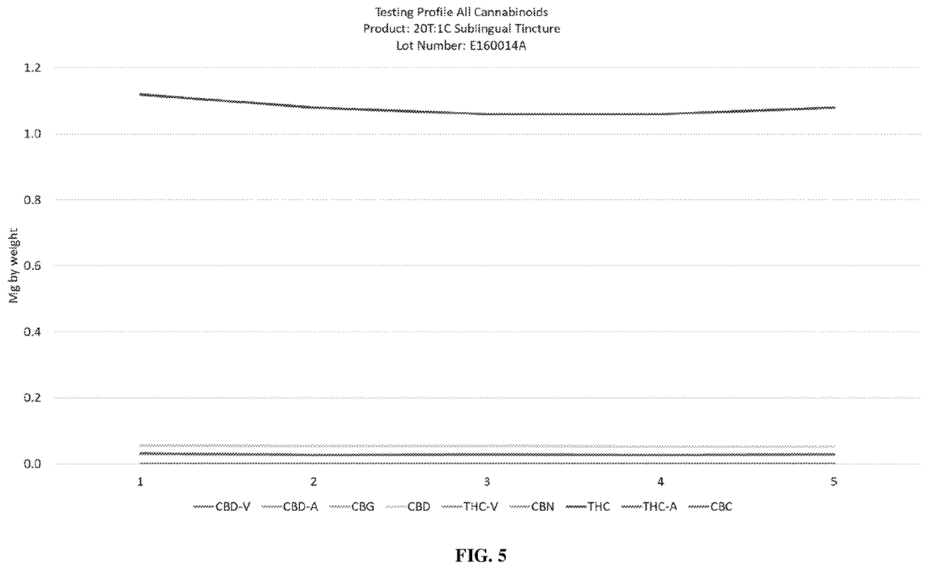

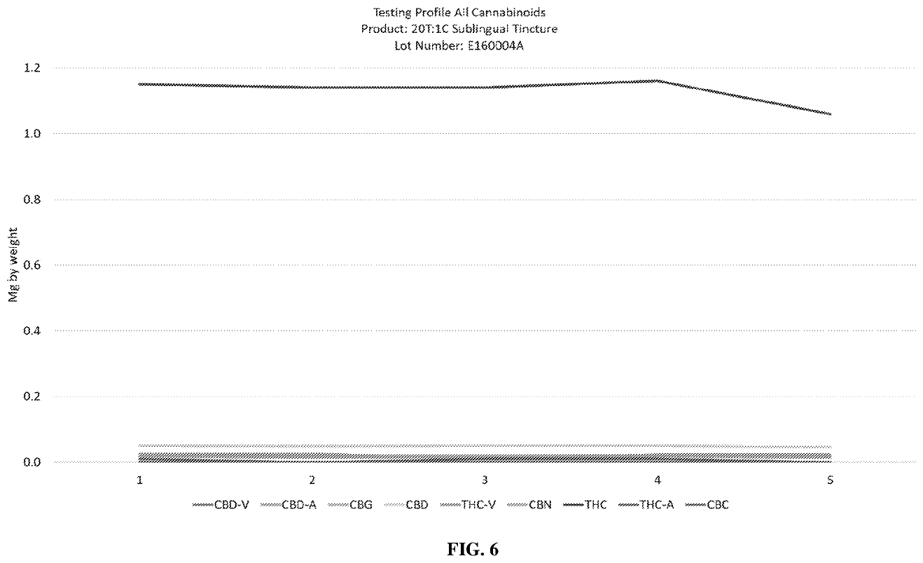

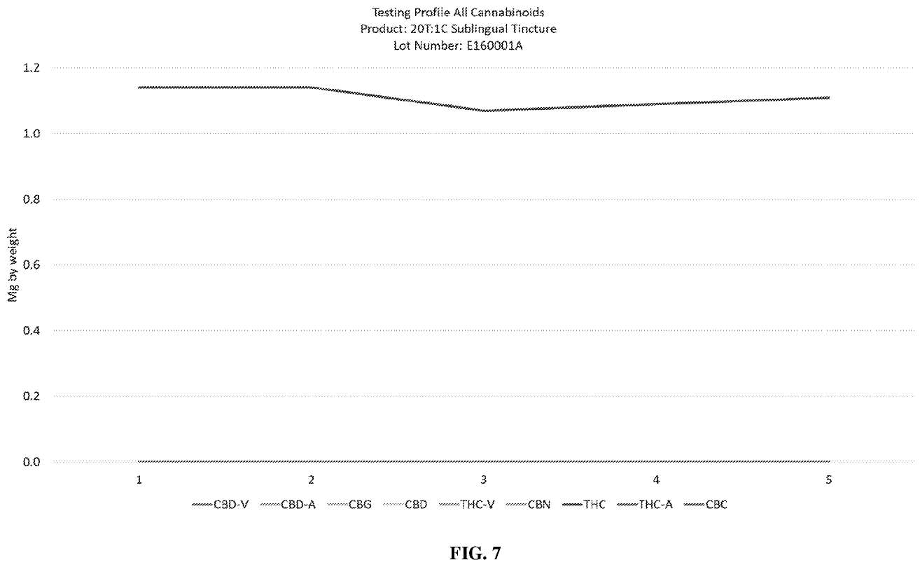

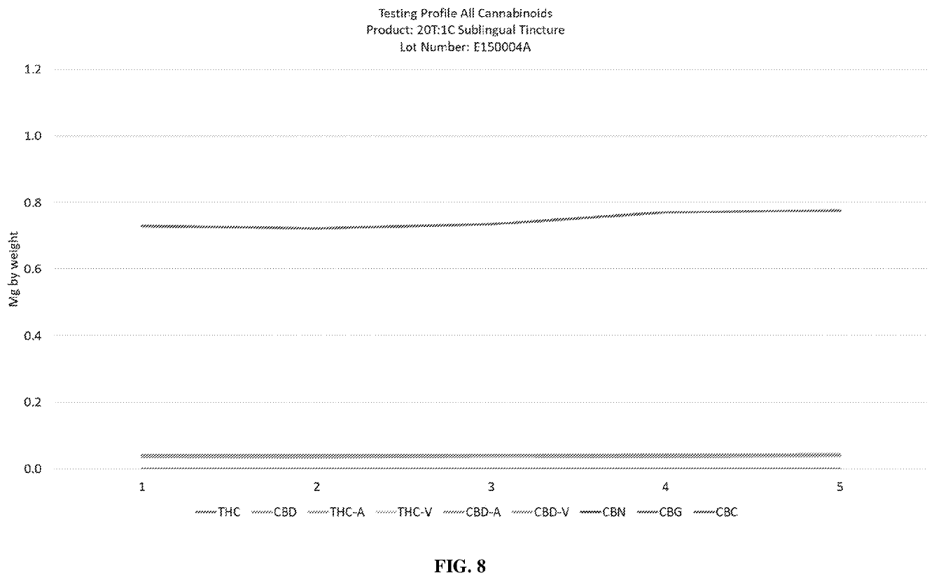

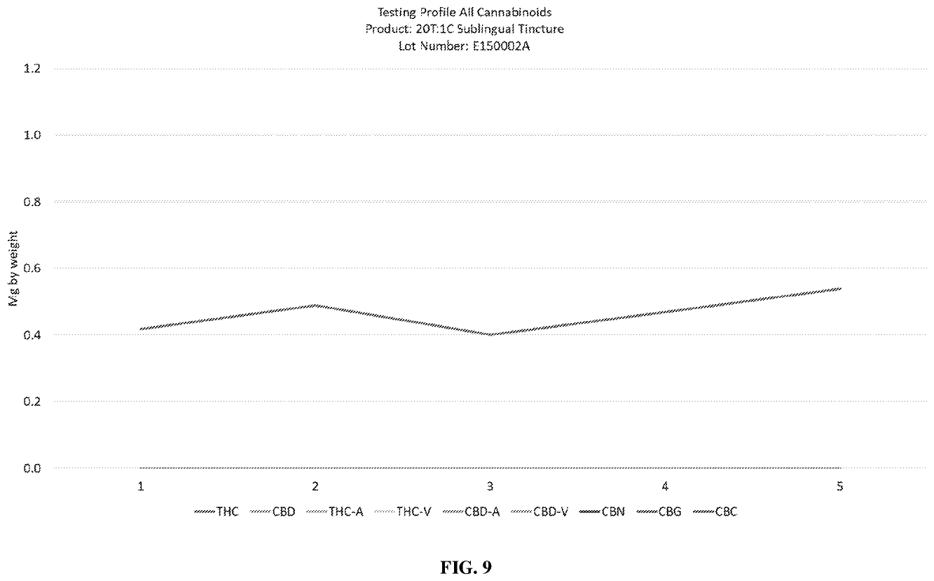

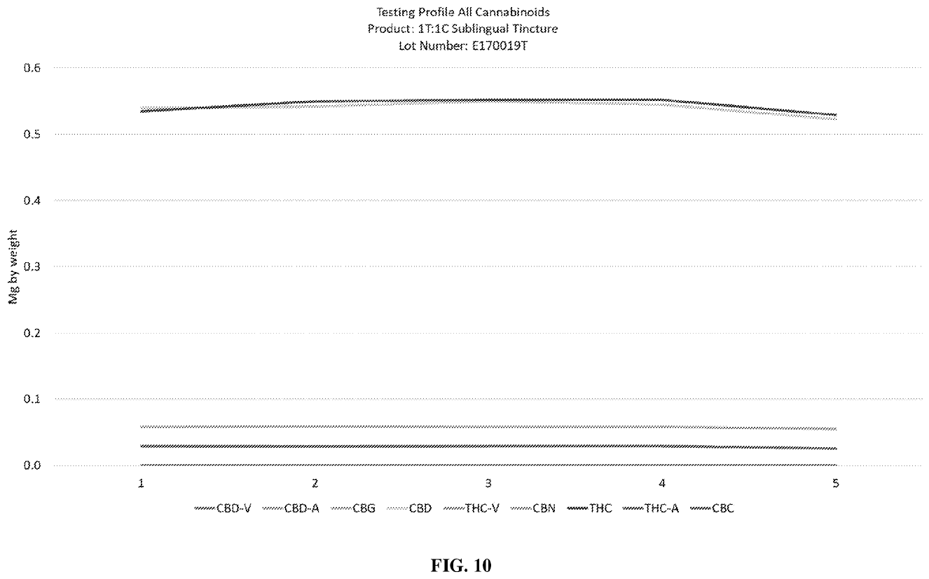

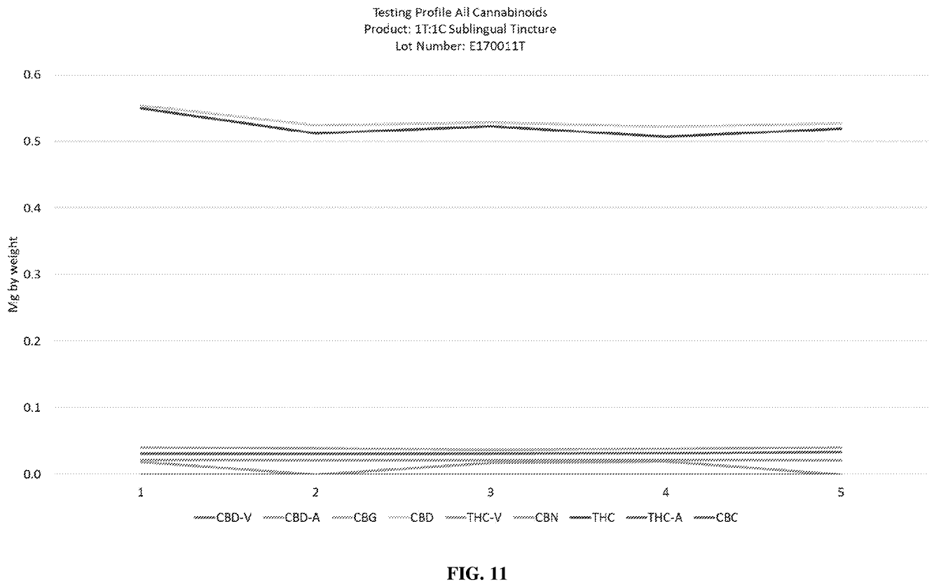

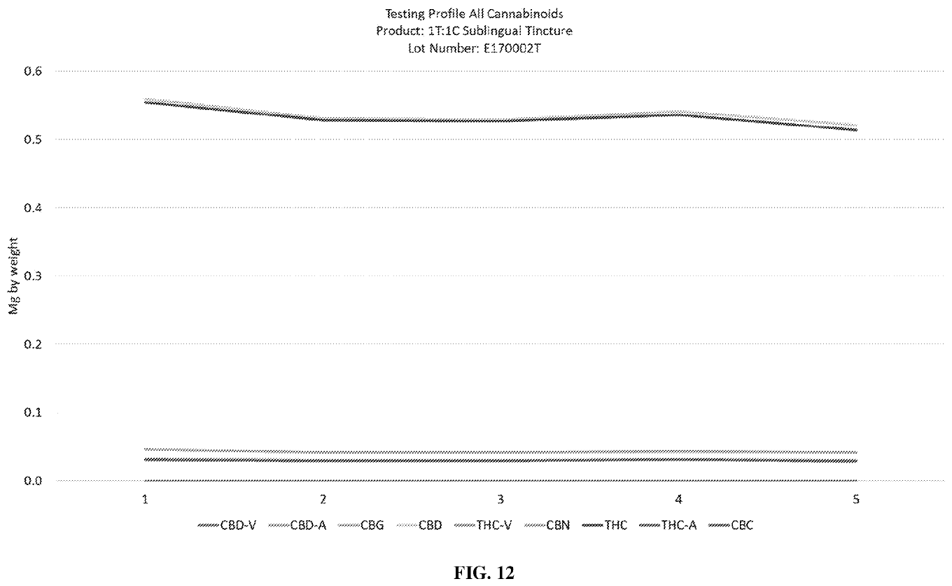

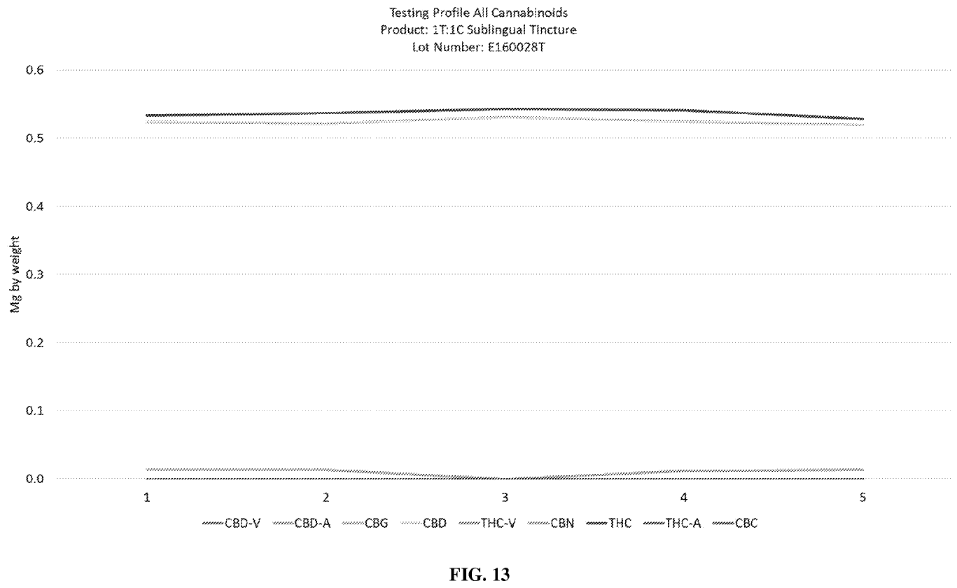

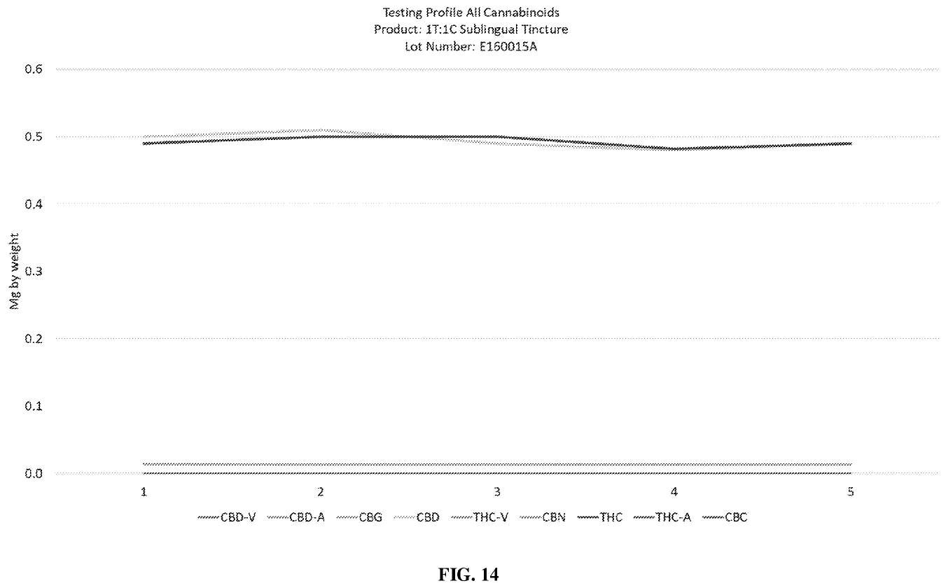

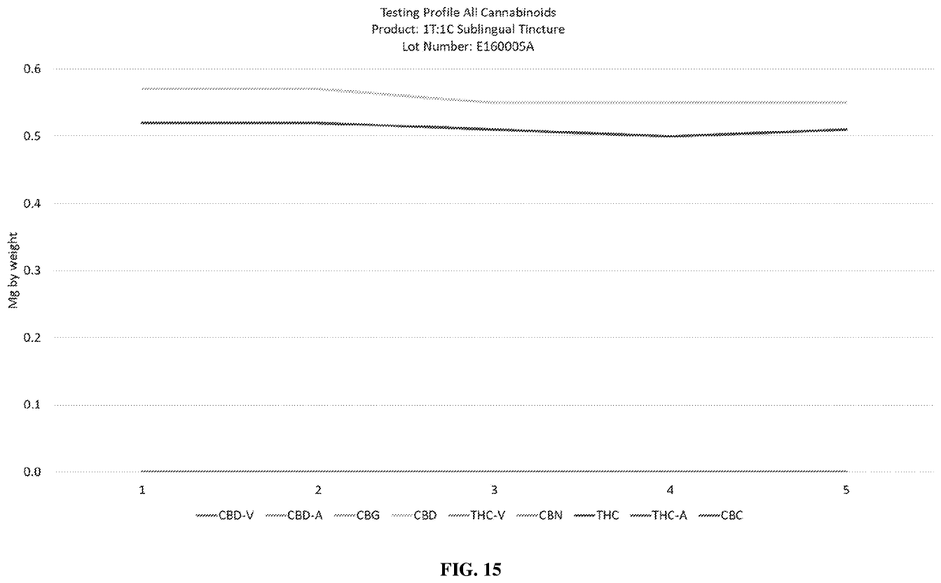

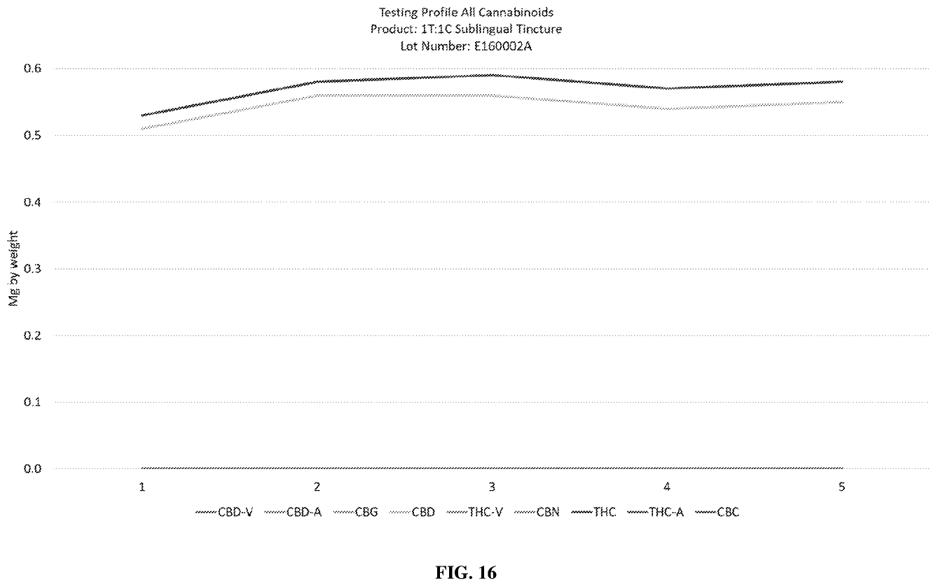

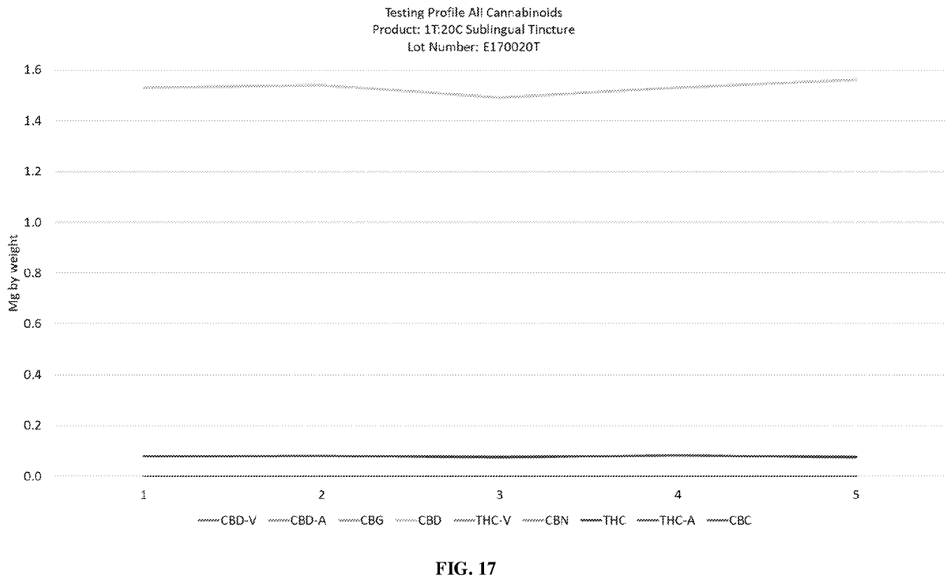

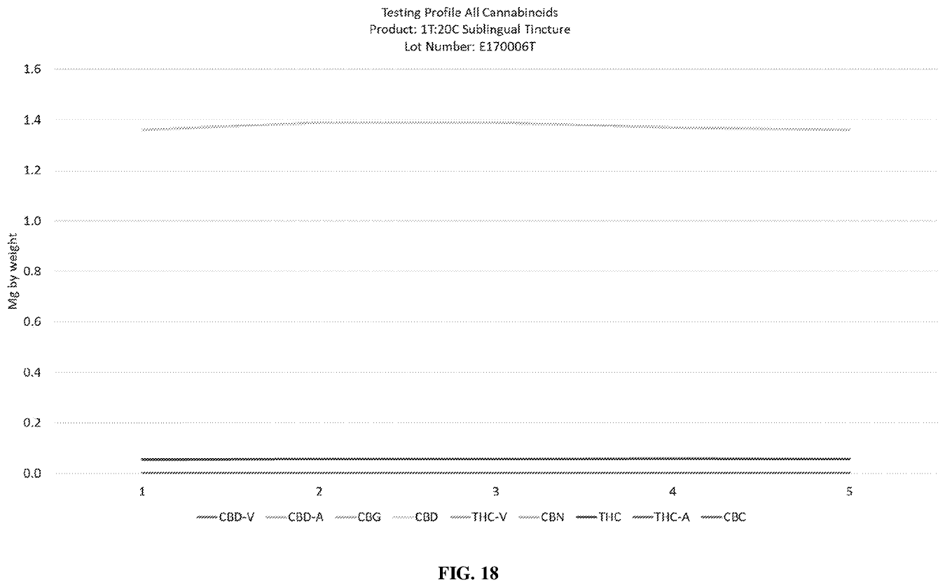

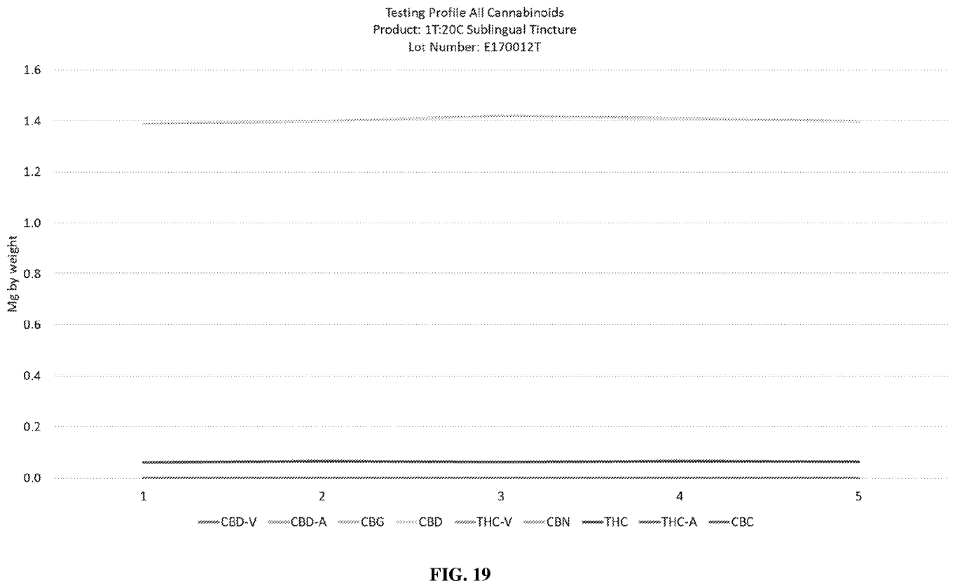

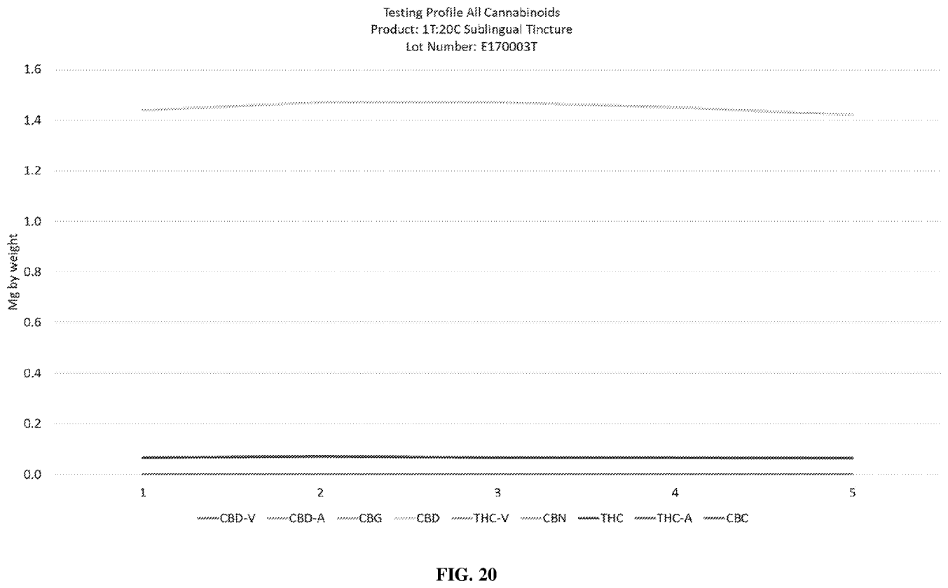

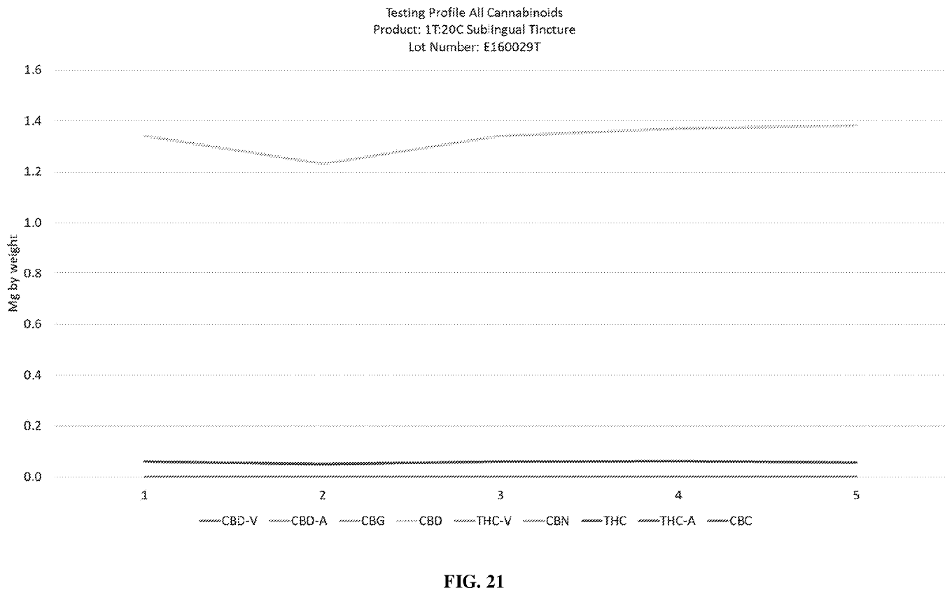

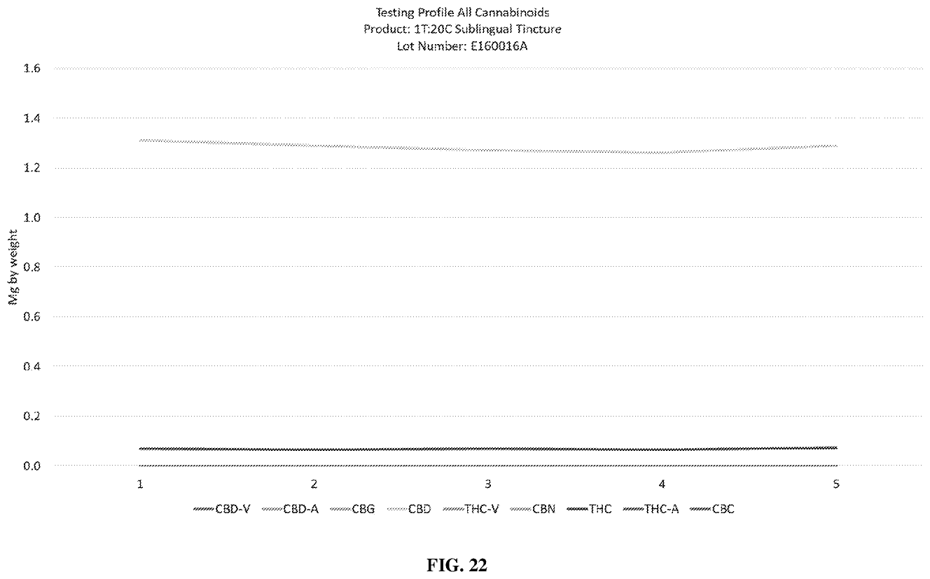

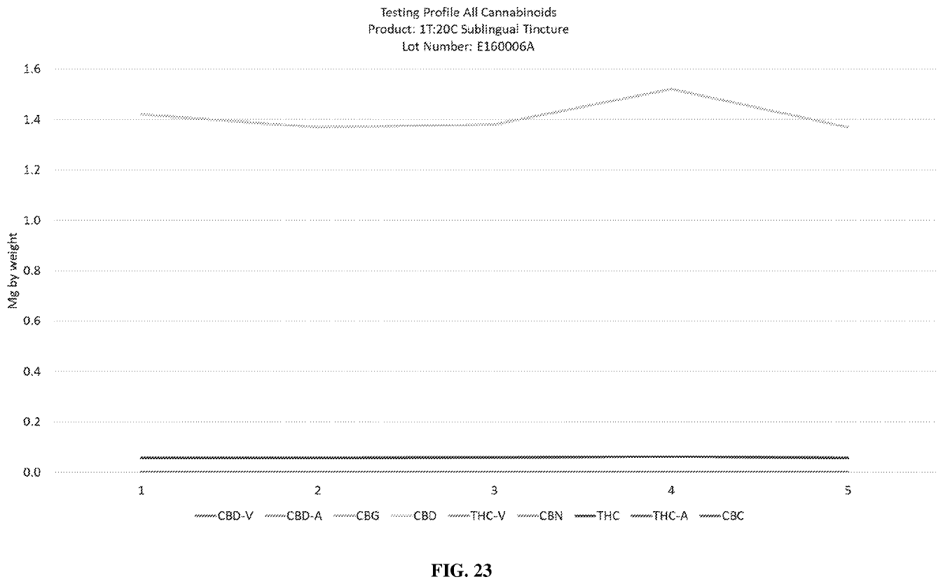

### DETAILED DESCRIPTION OF THE INVENTION

Before the present invention is disclosed and described, it is to be understood that this invention is not limited to the particular structures, process steps, or materials disclosed herein, but is extended to equivalents thereof as would be recognized by those of ordinarily skilled in the relevant arts. It should also be understood that terminology employed herein is used for the purpose of describing particular embodiments only and is not intended to be limiting.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, other embodiments includes from the one particular and/or to the other particular value.

Similarly, when values are expressed as approximations, by use of the antecedent “about,” it is understood that the particular value forms other embodiments. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” “medicament,” or “food product” are used interchangeably herein, as context dictates, to refer to a compound or compounds or composition of matter which, when administered to a subject (human or animal) induces a desired pharmacological and/or physiologic effect by local and/or systemic action.

The terms “subject,” “individual,” and “patient” are used interchangeably herein, and refer to an animal, for example a human, to whom treatment with a composition or formulation or food product in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The formulations described herein can be used to treat any suitable mammal, including primates, such as monkeys and humans, horses, cows, cats, dogs, rabbits, and rodents such as rats and mice. In some embodiments, the mammal to be treated is human. The human can be any human of any age. In some embodiments, the human is an adult. In some embodiments, the human can be male, female, middle-aged, adolescent, or elderly. According to any of the methods of the present invention and in some embodiments, the subject is human.

Conditions and disorders in a subject for which a particular drug, compound, composition, formulation, food product, dietary supplement (or combination thereof) is said herein to be “indicated” are not restricted to conditions and disorders for which that drug or compound or composition or formulation or food product or supplement has been expressly approved by a regulatory authority, but also include other conditions and disorders known or reasonably believed by a physician or other health or nutritional practitioner to be amenable to treatment with that drug or compound or composition or formulation or food product or supplement or combination thereof.

Standard conditions for cannabinoid assays, and methods of calculating cannabinoid content (as %) are well known in the art.

### Compositions

In certain aspects, the present invention is directed to a composition comprising: (a) at least one cannabinoid or cannabinoid extract; (b) triglyceride; (c) alcohol; and (d) at least one surfactant.

In certain embodiments, said triglyceride is an oil, such as a medium chain triglyceride (MCT). In some such embodiments, the MCT comprises a C-6 fatty acid, a C-8 fatty acid, a C-10 fatty acid, a C-12 fatty acid, or a combination thereof.

In certain embodiments, said alcohol is ethanol. In certain embodiments, said surfactant comprises polysorbate 80. In certain embodiments, said surfactant is polysorbate 80.

In certain embodiments, said surfactant comprises lecithin, such as soy lecithin. In certain embodiments, said surfactant is lecithin, such as soy lecithin.

In certain embodiments, the at least one cannabinoid or cannabinoid extract is selected from tetrahydrocannabinolic acid (THCa), cannabidiolic acid (CBDa), cannabinolic acid (CBNa), cannabichromenic acid (CBCa), tetrahydrocannabinol (THC), cannabinol (CBN), cannabidiol (CBD) and cannabichromene (CBC).

In certain embodiments, the composition has a combination of at least two cannabinoids. In certain embodiments, the composition comprises a combination of at least two cannabinoids. In certain embodiments, the two cannabinoids are selected from tetrahydrocannabinol (THC), cannabidiol (CBD), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cnnabichromevarin (CBCV), cannabigerovarin (CBGV), cannabigerol monomethyl ether (CBGM) and derivatives thereof, such as wherein the cannabinoids are THC and CBD or wherein the two cannabinoids are THCa and CBDa. In certain embodiments, the two cannabinoids are THC and CBD. In certain embodiments, two cannabinoids are THCa and CBDa.

In certain embodiments, the composition is in the form of a liquid or a gel.

In certain embodiments, at least two cannabinoids are present in a 1:1 proportion by weight. In other embodiments, at least two cannabinoids are present in a 10:1 proportion by weight. In still other embodiments, at least two cannabinoids are present in a 20:1 proportion by weight.

In certain embodiments, the total weight of one cannabinoid is about 5 mg/dose and the total weight of a second cannabinoid is about 5 mg/dose. In certain embodiments, the total weight of one cannabinoid is between 0.01 mg/dose and 0.5 mg/dose and the total weight of a second cannabinoid is between 9 mg/dose and 10 mg/dose. In certain embodiments, the total amount of one or more cannabinoids is between about 0.1 grams and about 10 grams.

In certain embodiments, at least one cannabinoid is THC and is present in an amount ranging from about 0.1 mg to about 10 mg. In other embodiments, at least one cannabinoid is CBD and is present in an amount ranging from about 0.1 mg to about 10 mg.

In certain embodiments, the total amount of one or more cannabinoids or cannabinoid extracts are present in an amount of about 1% to 2% by weight of the composition. In other embodiments, the total amount of one or more cannabinoids or cannabinoid extracts are present in an amount of about 1.5% by weight of the composition.

In some embodiments, a composition of the present invention is a pharmaceutical composition or formulation. In some such embodiments, said composition is formulated for penetrating a mucosal barrier.

In other such embodiments, said composition is formulated for rapid disintegration upon administration. In some embodiments, “rapid disintegration” means an in vitro disintegration time of 30 seconds or less, such as 25 seconds, 20 seconds, 15 seconds, 10 seconds, 5 seconds, 2 seconds, or 1 second, based on the U.S. Pharmacopeia disintegration test method or other methods known in the art.

In some embodiments, the composition/pharmaceutical composition is a unit dose of the composition/pharmaceutical composition. In some embodiments, the composition is formulated in a dosage form. In some embodiments, the dosage form is a unit dose. In some embodiments, the pharmaceutical composition is formulated in a unit dose. In some embodiments, the unit dose is for oral administration, i.e., an oral unit dosage form. In some embodiments, the unit dose is for sublingual (held under the tongue) or buccal (held between the cheek and gum) administration, i.e., a sublingual or buccal unit dosage form. In further embodiments, the unit dose is a liquid, solid, or semi-solid. In some such embodiments, the unit dose is a liquid. In some embodiments, the composition is a liquid. In other embodiments, the dosage form is a tincture.

The unit dose may be in the form of a tincture. In some embodiments, the dosage form is a tincture. In some embodiments, the tincture is an oral unit dosage form and, in some embodiments, the same is a sublingual or buccal unit dosage form.

In certain embodiments, at least two cannabinoids are present in the unit dose in a 20:1 proportion by weight. In other embodiments, at least two cannabinoids are present in a 10:1 proportion by weight. In still other embodiments, at least two cannabinoids are present in a 1:1 proportion by weight.

In certain embodiments, a first cannabinoid weighs between about 10 mg/ml and 10.4 mg/ml (e.g., of the tincture), and a second cannabinoid weighs between about 0.47 mg/ml and 0.6 mg/ml (e.g., of the tincture). In other embodiments, a first cannabinoid weighs about 0.56 mg/ml (e.g., of the tincture) and a second cannabinoid weighs about 12.6 mg/ml (e.g., of the tincture). In still other embodiments, a first cannabinoid weighs between about 4.9 mg/ml and 5.5 mg/ml (e.g., of the tincture) and a second cannabinoid weighs between about 4.9 mg/ml and 5.5 mg/ml (e.g., of the tincture).

In certain such embodiments, a first cannabinoid is THC and a second cannabinoid is CBD.

In certain embodiments, the tincture is contained in a bottle for periodic administration (e.g., daily, weekly, monthly). In some such embodiments, a first cannabinoid weighs between about 70 mg and 72.8 mg (e.g., of the dosage form or the unit dose) and a second cannabinoid weighs between about 3.29 mg and 4.2 mg (e.g., of the dosage form or the unit dose). In other such embodiments, a first cannabinoid weighs between about 34.3 mg and 38.5 mg (e.g., of the dosage form or the unit dose) and a second cannabinoid weighs between about 34.3 mg and 38.5 mg (e.g., of the dosage form or the unit dose). In still other such embodiments, a first cannabinoid weighs about 3.92 mg (e.g., of the dosage form or the unit dose) and a second cannabinoid weighs about 88.2 mg (e.g., of the dosage form or the unit dose). In certain embodiments, a first cannabinoid is THC and a second cannabinoid is CBD.

In certain embodiments, the dosage form contains from about 0.1 to about 10 mg of total cannabinoids or cannabinoid extracts. In certain embodiments, the unit dose contains from about 0.1 to about 10 mg of total cannabinoids or cannabinoid extracts.

In certain embodiments, the invention relates to oral, buccal, or sublingual compositions of cannabinoids to provide a release of a combination of Tetrahydrocannabinol (THC) and Cannabidiol (CBD). The cannabinoids are easily prepared and formulated to provide consistent therapeutically effective dosage forms from lot to lot.

Surfactants are important excipients frequently used as stabilizers and solubilizers. There are many commercially available surfactants. They have different properties and the same surfactant may have a wide range of applications. The pharmaceutical surfactants lecithin; phosphadylcholine fractions, poloxamer, sodium cholate and polysorbate 80 are well tolerated and non-toxic. They are unlikely to induce allergic reactions, hypersensitivity or cytokine production.

Lecithin is a naturally occurring mixture of the diglycerides of stearic, palmitic, and oleic acids, linked to the choline ester of phosphoric acid, commonly called phosphatidylcholine. Hydrogenated Lecithin is the product of controlled hydrogenation of Lecithin. Bilayers of these phospholipids in water may form liposomes, a spherical structure in which the acyl chains are inside and not exposed to the aqueous phase. Lecithin and Hydrogenated Lecithin are used in a large number of cosmetic formulations as skin conditioning agents-miscellaneous and as surfactant-emulsifying agents. Hydrogenated Lecithin is also used as a nonsurfactant suspending agent. Lecithin is virtually nontoxic in acute oral studies, short-term oral studies, and subchronic dermal studies in animals. Lecithin is not a reproductive toxicant, nor is it mutagenic in several assays. Fiume Z. Int J Toxicol. 2001; 20 Suppl 1:21-45.

Soy lecithin one of the most widely used food additives on the market today. It is used as an emulsifier. It helps to emulsify numerous foods, even unlikely emulsions such as chocolate. In chocolate, lecithin stabilizes the cocoa butter fat so it doesn't separate from the moisture, cocoa solids and dairy.

In some embodiments, the compositions of the present invention contain cannabinoid extracts that contain a combination of at least two of the following: tetrahydrocannabinol (THC), cannabidiol (CBD), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV) and cannabigerol monomethyl ether (CBGM) and derivatives thereof. The cannabinoid may be natural or synthetic.

In some embodiments, the compositions of the present invention contain at least two cannabinoids that are in a 20:1 proportion by weight. In some embodiments, the compositions of the present invention contain at least two cannabinoids that are in a 10:1 proportion by weight. In some embodiments, the compositions of the present invention contain at least two cannabinoids that are in a 1:1 proportion by weight.

In some embodiments, the cannabinoid extract is a mixture of cannabinoids and include terpenes and/or flavonoids.

The methods of making cannabinoids extract is well known in the art. The cannabis plants are grown, harvested, and the cannabinoids are extracted through, for example, a CO₂ extraction process.

In some embodiments, the base comprises a medium chain triglyceride (MCT). In some embodiments, the MCT comprises a C-6 fatty acid, a C-8 fatty acid, a C-10 fatty acid, a C-12 fatty acid, or a combination thereof.

In some embodiments, the liquid formulation of the extracts is infused in a mixture of medium chain triglyceride (MCT), ethanol and polysorbate 80 (tween 80).

In some embodiments, the terpene comprises beta-myrcene, limonene, beta caryopyllene, caryopyllene oxide, terpineol, citronellol, linalool, humulene, beta-amyrin, cycloartenol, or a combination thereof. Any other suitable terpene can also be employed in accordance with the compositions and methods described herein.

In some embodiments, it may be advantageous to decarboxylate the inactive (carboxylic acid form) cannabinoids in the extracts and formulations of the invention.

In some embodiments, the total weight of one cannabinoid is between 0.01 mg/dose and 300 mg/dose and the total weight of a second cannabinoid is between 0.01 mg/dose and 300 mg/dose. In some embodiments, the total weight of one cannabinoid is between 0.01 mg/dose and 200 mg/dose and the total weight of a second cannabinoid is between 0.01 mg/dose and 200 mg/dose. In some embodiments, the total weight of one cannabinoid is between 0.01 mg/dose and 100 mg/dose and the total weight of a second cannabinoid is between 0.01 mg/dose and 100 mg/dose. In some embodiments, the total weight of one cannabinoid is between 0.01 mg/dose and 50 mg/dose and the total weight of a second cannabinoid is between 0.01 mg/dose and 50 mg/dose. In some embodiments, the total weight of one cannabinoid is between 0.01 mg/dose and 20 mg/dose and the total weight of a second cannabinoid is between 0.01 mg/dose and 20 mg/dose.

In some embodiments, the unit dose comprises about 0.25-100 mg of at least one active ingredient, e.g., cannabinoid or cannabinoid extract. In some embodiments, the unit dose comprises about 0.25-0.5 mg of at least one active ingredient, e.g., cannabinoid or cannabinoid extract. In some embodiments, the unit dose comprises about 0.5-1 mg of at least one active ingredient, e.g., cannabinoid or cannabinoid extract. In some embodiments, the unit dose comprises about 1-2.5 mg of at least one active ingredient, e.g., cannabinoid or cannabinoid extract. In some embodiments, the unit dose comprises about 2.5-5 mg of at least one active ingredient, e.g., cannabinoid or cannabinoid extract. In some embodiments, the unit dose comprises about 5-7.5 mg of at least one active ingredient, e.g., cannabinoid or cannabinoid extract. In some embodiments, the unit dose comprises about 7.5-10 mg of at least one active ingredient, e.g., cannabinoid or cannabinoid extract. In some embodiments, the unit dose comprises about 10 mg of at least one active ingredient, e.g., cannabinoid or cannabinoid extract.

In some embodiments, the unit dose comprises between 0.25 mg and 300 mg of total active ingredient comprising one or more cannabinoid or cannabinoid extract. In some embodiments, the unit dose comprises between 100 mg and 200 mg of total active ingredient comprising one or more cannabinoid or cannabinoid extract.

In some embodiments, the composition of present invention comprises between 0.25 mg and 300 mg of total active ingredient comprising one or more cannabinoid or cannabinoid extract. In some embodiments, the composition of present invention comprises between 100 mg and 200 mg of total active ingredient comprising one or more cannabinoid or cannabinoid extract.

The compositions disclosed herein include a composition for daily administration. In some embodiments, the therapeutic effect is maintained with one administration, twice daily. The duration of each dose's effect is between four (4) to six (6) hours.

In certain aspects, the present invention is directed to a method of sublingually or buccally administering a therapeutic agent to a patient, comprising the steps of: a) preparing a pharmaceutical composition as defined herein, and b) administering the pharmaceutical composition to the underside of the tongue of the patient. In certain embodiments, the pharmaceutical composition is a liquid.

In other aspects, the present invention is directed to a method for treating a disease comprising administering a daily therapeutically effective amount of the pharmaceutical composition as described herein or the dosage form as described herein.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. In some embodiments, by “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

Examples of a disease or a disorder that can be treated by the invention include, but not limited to, pain associated with cancer, neuropathic pain and HIV-associated sensory neuropathy, side effects of chemotherapy including nausea and pain, symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia. In certain embodiments, the disease is selected from pain associated with cancer, neuropathic pain and HIV-associated sensory neuropathy, side effects of chemotherapy including nausea and pain, symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia.

In some embodiments where the disorder is cancer, pain associated with cancer; nausea associated with chemotherapy; or a combination thereof, the composition described herein exerts reduced hallucinatory effects compared to smoking a cannabis containing cigarette or ingesting a cannabis containing foodstuff with the same amount of active ingredients.

The composition of the present invention may also contain additional ingredients such as solvents, carriers or excipients.

In some embodiments, the solvent comprises ethanol, methanol, isopropanol, chloroform, propylene glycol, polyethylene glycol, glycerine, limonene, myrcene, linalool, alpha bisabolol, delta 3 carene, borneol, alpha-pinene, beta-pinene, eucalyptol, terpineol, caryophyllene, camphene, or combinations thereof.

In some embodiments, cannabinoid of the invention is any member of a group of substances that are structurally related to tetrahydrocannabinol and that bind to a cannabinoid receptor such as CB1 or CB2 or both THC′). The cannabinoid can be a naturally occurring compound (e.g. present in Cannabis), a compound metabolized by a plant or animal, or a synthetic derivative.

The cannabinoid may be included in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; enol forms.

The cannabinoids of the invention are further meant to encompass natural cannabinoids, natural cannabinoids that have been purified or modified, and synthetically derived cannabinoids, for example, United States Patent Application Publication 2005/0266108, which is hereby incorporated by reference in its entirety, describes a method of purifying cannabinoids obtained from plant material.

The cannabinoids of the invention can be any of 9-tetrahydrocannabinol, 8-tetrahydrocannabinol, (+)-1,1-dimethylheptyl analog of 7-hydroxy-delta-6-tetrahydrocannabinol, 3-(5′-cyano-1′,1′-dimethylpentyl)-1-(4-N-morpholinobutyryloxy) delta 8-tetrahydrocannabinol hydrochloride], dexanabinol, nabilone, levonantradol, or N-(2-hydroxyethyl)hexadecanoamide. In some embodiments, the cannabinoids of the invention can be any of the non-psychotropic cannabinoid 3-dimethylnepty 11 carboxylic acid homologine 8, delta-8-tetrahydrocannabinol.

The above ingredients may be used by combining two or more members at an appropriate ratio.

All patents and literature references cited in the present specification are hereby incorporated by reference in their entirety.

The present invention will be specifically explained by way of examples, but these examples are not intended to limit the present invention.

### Example 1

### Sublingual and Buccal Dosage Forms of Cannabinoid Extracts

Exemplary formulations described above are shown in Table 1 and are prepared using the methods described herein. Exemplary formulations as described above and throughout comprise at least one cannabinoid, medium chain triglycerides, ethanol, and polysorbate 80.

TABLE 1
Exemplary Sublingual/Buccal Formulation

| Ingredient | Percentage by weight |
|---|---|
| Cannabinoid Extract | 1.5 |
| Medium Chain Triglycerides | 87.6 |
| Ethanol | 10.0 |
| Polysorbate 80 | 0.3 |
| Total | 100 |

It is to be understood that while the present example relates the composition having certain THC/CBD ratios, other THC/CBD ratios (e.g. 1:10, 10:1, etc.) are also encompassed by the invention and can be easily prepared using methods known in the art.

### Example 2

### Cannabinoid Profile of Sublingual and Buccal Tinctures

### Materials and Methods

Representative samples of the pharmaceutical formulations in tinctures were diluted/dissolved with organic solvents. A portion of formulation, typically from 10 to 1200 mg, were weighed into a 50-mL centrifuge tube. The amount weighed depended upon the specific product and the declared concentrations of cannabinoids. A Surrogate Standard (SUR) (a pure analyte, which should not be found in any sample, but is similar in nature to the compounds of interest) and 20.0-mL of methanol (MeOH) were added. The solution was mixed well and was either diluted further or used directly for analysis. If necessary, this extract was diluted an additional 2- to 20-fold based on the concentrations of cannabinoids in the sample. The internal standard working diluent (IWD) (a solution of internal standard that is prepared from the internal standard stock diluent and added to all samples at the same concentration) was then added to the extract or dilution thereof, and the potency measurement was made using HPLC-PDA.

The diluted samples fortified with internal standard were injected onto an HPLC. The targeted analytes were separated and subsequently detected online by monitoring UV absorbance using a PDA detector. The separation of ten cannabinoids was achieved on a C18 reverse-phase column 150 mm in length. The limit of quantification for most of the cannabinoids was approximately 0.60 i.t.g/mL. This method was used to quantify the cannabinoid components that are present as low as 0.04% (percent by weight) in the formulations.

Tables 1-9 show the cannabinoid profile for pharmaceutical formulations in tinctures containing two cannabinoids in a ratio of 20:1 THC to CBD by weight.

TABLE 1
Lot Number E170018T
20 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.029 | 0.030 | 0.031 | 0.030 | 0.028 | 0.030 |
| CBD | 0.054 | 0.052 | 0.055 | 0.055 | 0.051 | 0.053 |
| THC-V | 0.000 | 0.000 | 0.000 | 0.000 | 0.000 | 0.000 |
| CBN | 0.020 | 0.020 | 0.019 | 0.019 | 0.019 | 0.019 |
| THC | 1.030 | 1.010 | 1.040 | 1.060 | 1.020 | 1.032 |
| THC-A | 0.000 | 0.000 | 0.000 | 0.000 | 0.000 | 0.000 |
| CBC | 0.000 | 0.000 | 0.000 | 0.000 | 0.000 | 0.000 |

TABLE 2
Lot Number E170017T
20 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.031 | 0.032 | 0.031 | 0.030 | 0.031 | 0.031 |
| CBD | 0.067 | 0.065 | 0.065 | 0.067 | 0.066 | 0.066 |
| THC-V | 0.000 | 0.000 | 0.000 | 0.000 | 0.000 | 0.000 |
| CBN | 0.037 | 0.039 | 0.039 | 0.039 | 0.039 | 0.039 |
| THC | 1.100 | 1.150 | 1.110 | 1.140 | 1.110 | 1.122 |
| THC-A | 0.000 | 0.000 | 0.000 | 0.000 | 0.000 | 0.000 |
| CBC | 0.019 | 0.020 | 0.021 | 0.020 | 0.020 | 0.020 |

TABLE 3
Lot Number E170001T
20 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.032 | 0.033 | 0.031 | 0.031 | 0.031 | 0.032 |
| CBD | 0.058 | 0.061 | 0.056 | 0.057 | 0.056 | 0.058 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 1.060 | 1.070 | 1.040 | 1.050 | 1.040 | 1.052 |
| THC-A | 0.083 | 0.083 | 0.081 | 0.084 | 0.086 | 0.083 |
| CBC | 0.000 | 0.000 | 0.000 | 0.000 | 0.000 | 0.000 |

TABLE 4
Lot Number E160027T
20 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.030 | 0.030 | 0.029 | 0.030 | 0.031 | 0.030 |
| CBD | 0.056 | 0.056 | 0.056 | 0.056 | 0.056 | 0.056 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 1.070 | 1.100 | 1.120 | 1.070 | 1.080 | 1.088 |
| THC-A | 0.000 | 0.000 | 0.000 | 0.000 | 0.000 | 0.000 |
| CBC | 0.000 | 0.000 | 0.000 | 0.000 | 0.000 | 0.000 |

TABLE 5
Lot Number E160014A
20 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.030 | 0.026 | 0.027 | 0.026 | 0.028 | 0.027 |
| CBD | 0.055 | 0.054 | 0.053 | 0.052 | 0.052 | 0.053 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 1.120 | 1.080 | 1.060 | 1.060 | 1.080 | 1.080 |
| THC-A | 0.032 | 0.026 | 0.028 | 0.026 | 0.028 | 0.028 |
| CBC | 0.000 | 0.000 | 0.000 | 0.000 | 0.000 | 0.000 |

TABLE 6
Lot Number E 160004A
20 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.025 | 0.026 | 0.010 | 0.023 | 0.023 | 0.021 |
| CBD | 0.049 | 0.048 | 0.049 | 0.049 | 0.045 | 0.048 |
| THC-V | 0.020 | 0.020 | 0.019 | 0.021 | 0.020 | 0.020 |
| CBN | 0.016 | 0.014 | 0.015 | 0.016 | 0.014 | 0.015 |
| THC | 1.150 | 1.140 | 1.140 | 1.160 | 1.060 | 1.130 |
| THC-A | 0.000 | 0.000 | 0.000 | 0.000 | 0.000 | 0.000 |
| CBC | 0.009 | 0.000 | 0.010 | 0.010 | 0.000 | 0.006 |

TABLE 7
Lot Number E160001A
20 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.000 | 0.000 | 0.000 | 0.000 | 0.000 | 0.000 |
| CBD | 0.000 | 0.000 | 0.000 | 0.000 | 0.000 | 0.000 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 1.140 | 1.140 | 1.070 | 1.090 | 1.110 | 1.110 |
| THC-A | 0.000 | 0.000 | 0.000 | 0.000 | 0.000 | 0.000 |
| CBC | 0.000 | 0.000 | 0.000 | 0.000 | 0.000 | 0.000 |

TABLE 8
Lot Number E150004A
20 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| THC | 0.729 | 0.722 | 0.735 | 0.771 | 0.776 | 0.747 |
| CBD | 0.041 | 0.040 | 0.041 | 0.042 | 0.044 | 0.042 |
| THC-A | 0.035 | 0.034 | 0.036 | 0.035 | 0.038 | 0.036 |
| THC-V | 0.000 | 0.000 | 0.000 | 0.000 | 0.000 | 0.000 |
| CBD-A | 0.000 | 0.000 | 0.000 | 0.000 | 0.000 | 0.000 |
| CBD-V | 0.000 | 0.000 | 0.000 | 0.000 | 0.000 | 0.000 |
| CBN | 0.000 | 0.000 | 0.000 | 0.000 | 0.000 | 0.000 |
| CBG | 0.000 | 0.000 | 0.000 | 0.000 | 0.000 | 0.000 |
| CBC | 0.000 | 0.000 | 0.000 | 0.000 | 0.000 | 0.000 |

TABLE 9
Lot Number E150002A
20 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| THC | 0.418 | 0.489 | 0.401 | 0.469 | 0.539 | 0.463 |
| CBD | 0.000 | 0.000 | 0.000 | 0.000 | 0.000 | 0.000 |
| THC-A | 0.000 | 0.000 | 0.000 | 0.000 | 0.000 | 0.000 |
| THC-V | 0.000 | 0.000 | 0.000 | 0.000 | 0.000 | 0.000 |
| CBD-A | 0.000 | 0.000 | 0.000 | 0.000 | 0.000 | 0.000 |
| CBD-V | 0.000 | 0.000 | 0.000 | 0.000 | 0.000 | 0.000 |
| CBN | 0.000 | 0.000 | 0.000 | 0.000 | 0.000 | 0.000 |
| CBG | 0.000 | 0.000 | 0.000 | 0.000 | 0.000 | 0.000 |
| CBC | 0.000 | 0.000 | 0.000 | 0.000 | 0.000 | 0.000 |

Tables 10-16 show the cannabinoid profile for pharmaceutical formulations in tinctures containing two cannabinoids in a ratio of 1:1 THC to CBD by weight.

TABLE 10
Lot Number E170019T
1 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.058 | 0.059 | 0.058 | 0.058 | 0.055 | 0.058 |
| CBD | 0.540 | 0.542 | 0.550 | 0.545 | 0.523 | 0.540 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.535 | 0.550 | 0.552 | 0.552 | 0.529 | 0.544 |
| THC-A | 0.000 | 0.000 | 0.000 | 0.000 | 0.000 | 0.000 |
| CBC | 0.029 | 0.028 | 0.029 | 0.029 | 0.025 | 0.028 |

TABLE 11
Lot Number E170011T
1 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.022 | 0.021 | 0.021 | 0.022 | 0.021 | 0.021 |
| CBG | 0.040 | 0.039 | 0.037 | 0.038 | 0.040 | 0.039 |
| CBD | 0.554 | 0.524 | 0.528 | 0.522 | 0.527 | 0.531 |
| THC-V | 0.000 | 0.000 | 0.000 | 0.000 | 0.000 | 0.000 |
| CBN | 0.018 | 0.000 | 0.017 | 0.019 | 0.000 | 0.011 |
| THC | 0.550 | 0.512 | 0.523 | 0.507 | 0.519 | 0.522 |
| THC-A | 0.000 | 0.000 | 0.000 | 0.000 | 0.000 | 0.000 |
| CBC | 0.031 | 0.030 | 0.031 | 0.032 | 0.033 | 0.031 |

TABLE 12
Lot Number E170002T
1 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.032 | 0.030 | 0.030 | 0.031 | 0.030 | 0.031 |
| CBG | 0.046 | 0.042 | 0.042 | 0.043 | 0.042 | 0.043 |
| CBD | 0.560 | 0.531 | 0.529 | 0.541 | 0.521 | 0.536 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.555 | 0.528 | 0.527 | 0.536 | 0.514 | 0.532 |
| THC-A | 0.000 | 0.000 | 0.000 | 0.000 | 0.000 | 0.000 |
| CBC | 0.030 | 0.029 | 0.029 | 0.031 | 0.028 | 0.029 |

TABLE 13
Lot Number E160028T
1 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.013 | 0.013 | 0.000 | 0.012 | 0.013 | 0.010 |
| CBD | 0.524 | 0.522 | 0.531 | 0.525 | 0.520 | 0.524 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.533 | 0.537 | 0.543 | 0.541 | 0.528 | 0.536 |
| THC-A | 0.000 | 0.000 | 0.000 | 0.000 | 0.000 | 0.000 |
| CBC | 0.000 | 0.000 | 0.000 | 0.000 | 0.000 | 0.000 |

TABLE 14
Lot Number E160015A 1 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.014 | 0.013 | 0.013 | 0.013 | 0.013 | 0.013 |
| CBD | 0.500 | 0.510 | 0.490 | 0.480 | 0.490 | 0.494 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.490 | 0.500 | 0.500 | 0.482 | 0.490 | 0.492 |
| THC-A | 0.000 | 0.000 | 0.000 | 0.000 | 0.000 | 0.000 |
| CBC | 0.000 | 0.000 | 0.000 | 0.000 | 0.000 | 0.000 |

TABLE 15
Lot Number E160005A 1 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.000 | 0.000 | 0.000 | 0.000 | 0.000 | 0.000 |
| CBD | 0.570 | 0.570 | 0.550 | 0.550 | 0.550 | 0.558 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.520 | 0.520 | 0.510 | 0.500 | 0.510 | 0.512 |
| THC-A | 0.000 | 0.000 | 0.000 | 0.000 | 0.000 | 0.000 |
| CBC | 0.000 | 0.000 | 0.000 | 0.000 | 0.000 | 0.000 |

TABLE 16
Lot Number E 160002A
1 THC: 1 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.000 | 0.000 | 0.000 | 0.000 | 0.000 | 0.000 |
| CBD | 0.510 | 0.560 | 0.560 | 0.540 | 0.550 | 0.544 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.530 | 0.580 | 0.590 | 0.570 | 0.580 | 0.570 |
| THC-A | 0.000 | 0.000 | 0.000 | 0.000 | 0.000 | 0.000 |
| CBC | 0.000 | 0.000 | 0.000 | 0.000 | 0.000 | 0.000 |

Tables 17-23 show the cannabinoid profile for pharmaceutical formulations in tinctures containing two cannabinoids in a ratio of 1:20 THC to CBD by weight.

TABLE 17
Lot Number E170020T
1 THC: 20 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.000 | 0.000 | 0.000 | 0.000 | 0.000 | 0.000 |
| CBD | 1.530 | 1.540 | 1.490 | 1.530 | 1.560 | 1.530 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.078 | 0.080 | 0.076 | 0.083 | 0.076 | 0.079 |
| THC-A | 0.000 | 0.000 | 0.000 | 0.000 | 0.000 | 0.000 |
| CBC | 0.000 | 0.000 | 0.000 | 0.000 | 0.000 | 0.000 |

TABLE 18
Lot Number E170006T
1 THC: 20 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.000 | 0.000 | 0.000 | 0.000 | 0.000 | 0.000 |
| CBD | 1.360 | 1.390 | 1.390 | 1.370 | 1.360 | 1.374 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.055 | 0.560 | 0.056 | 0.058 | 0.057 | 0.157 |
| THC-A | 0.000 | 0.000 | 0.000 | 0.000 | 0.000 | 0.000 |
| CBC | 0.000 | 0.000 | 0.000 | 0.000 | 0.000 | 0.000 |

TABLE 19
Lot Number E170012T
1 THC: 20 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.000 | 0.000 | 0.000 | 0.000 | 0.000 | 0.000 |
| CBD | 1.390 | 1.400 | 1.420 | 1.410 | 1.400 | 1.404 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.060 | 0.065 | 0.061 | 0.065 | 0.063 | 0.063 |
| THC-A | 0.000 | 0.000 | 0.000 | 0.000 | 0.000 | 0.000 |
| CBC | 0.000 | 0.000 | 0.000 | 0.000 | 0.000 | 0.000 |

TABLE 20
Lot Number E170003T
1 THC: 20 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.000 | 0.000 | 0.000 | 0.000 | 0.000 | 0.000 |
| CBD | 1.440 | 1.470 | 1.470 | 1.450 | 1.420 | 1.450 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.065 | 0.072 | 0.065 | 0.066 | 0.064 | 0.066 |
| THC-A | 0.000 | 0.000 | 0.000 | 0.000 | 0.000 | 0.000 |
| CBC | 0.000 | 0.000 | 0.000 | 0.000 | 0.000 | 0.000 |

TABLE 21
Lot Number E 160029T
1 THC: 20 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.000 | 0.000 | 0.000 | 0.000 | 0.000 | 0.000 |
| CBD | 1.340 | 1.230 | 1.340 | 1.370 | 1.380 | 1.332 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.059 | 0.050 | 0.060 | 0.062 | 0.057 | 0.058 |
| THC-A | 0.000 | 0.000 | 0.000 | 0.000 | 0.000 | 0.000 |
| CBC | 0.000 | 0.000 | 0.000 | 0.000 | 0.000 | 0.000 |

TABLE 22
Lot Number E 160016A
1 THC: 20 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.000 | 0.000 | 0.000 | 0.000 | 0.000 | 0.000 |
| CBD | 1.310 | 1.290 | 1.270 | 1.260 | 1.290 | 1.284 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.069 | 0.066 | 0.069 | 0.065 | 0.073 | 0.068 |
| THC-A | 0.000 | 0.000 | 0.000 | 0.000 | 0.000 | 0.000 |
| CBC | 0.000 | 0.000 | 0.000 | 0.000 | 0.000 | 0.000 |

TABLE 23
Lot Number E 160006A
1 THC: 20 CBD Tincture

| | Percent Cannabinoid by Weight | | | | | |
|---|---|---|---|---|---|---|
| | Sam- | Sam- | Sam- | Sam- | Sam- | |
| Cannabinoid | ple 1 | ple 2 | ple 3 | ple 4 | ple 5 | Average |
| CBD-V | 0.000 | 0.000 | 0.000 | 0.000 | 0.000 | 0.000 |
| CBD-A | 0.000 | 0.000 | 0.000 | 0.000 | 0.000 | 0.000 |
| CBG | 0.000 | 0.000 | 0.000 | 0.000 | 0.000 | 0.000 |
| CBD | 1.420 | 1.370 | 1.380 | 1.520 | 1.370 | 1.412 |
| THC-V | 0.000 | 0.000 | 0.000 | 0.000 | 0.000 | 0.000 |
| CBN | 0.000 | 0.000 | 0.000 | 0.000 | 0.000 | 0.000 |
| THC | 0.056 | 0.056 | 0.058 | 0.062 | 0.057 | 0.058 |
| THC-A | 0.000 | 0.000 | 0.000 | 0.000 | 0.000 | 0.000 |
| CBC | 0.000 | 0.000 | 0.000 | 0.000 | 0.000 | 0.000 |

Unless defined otherwise, all technical and scientific terms used herein have the same meanings as commonly understood by one of ordinary skill in the art.

Having described preferred embodiments of the present invention with reference to the accompanying drawings, it is to be understood that the present invention is not limited to the above-mentioned embodiments and that various changes and modifications can be affected by one skilled in the art without departing from the spirit or scope of the present invention as defined in the appended claims.

What is claimed is:

1. A composition comprising:
(a) at least one cannabinoid or cannabinoid extract;
(b) triglyceride;
(c) alcohol; and
(d) at least one surfactant.

2. The composition of claim 1, wherein said triglyceride is an oil.

3. The composition of any one of the preceding claims, wherein said triglyceride is a medium chain triglyceride (MCT).

4. The composition of claim 3, wherein said MCT comprises a C-6 fatty acid, a C-8 fatty acid, a C-10 fatty acid, a C-12 fatty acid, or a combination thereof.

5. The composition of any one of the preceding claims, wherein said alcohol is ethanol.

6. The composition of any one of the preceding claims, wherein said surfactant is polysorbate 80.

7. The composition of any one claims 1-5, wherein said surfactant is lecithin.

8. The composition of claim 7, wherein said lecithin is soy lecithin.

9. The composition of any one of the preceding claims, wherein the at least one cannabinoid or canninboid extract is selected from the group consisting of tetrahydrocannabinolic acid (THCa), cannabidiolic acid (CBDa), cannabinolic acid (CBNa), cannabichromenic acid (CBCa), tetrahydrocannabinol (THC), cannabinol (CBN), cannabidiol (CBD) and cannabichromene (CBC).

10. The composition of any one of the preceding claims, wherein the composition has a combination of at least two cannabinoids.

11. The composition of claim 10, wherein the two cannabinoids are selected from tetrahydrocannabinol (THC), cannabidiol (CBD), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cnnabichromevarin (CBCV), cannabigerovarin (CBGV), cannabigerol monomethyl ether (CBGM) and derivatives thereof.

12. The composition of claim 10, wherein said cannabinoids are THC and CBD.

13. The composition of claim 10, wherein the two cannabinoids are THCa and CBDa.

14. The composition of any one of the preceding claims, wherein the composition is in the form of a liquid or a gel.

15. The composition of any one of claims 1-14, wherein at least two cannabinoids are in a 1:1 proportion by weight.

16. The composition of any one of claims 1-14, wherein the total weight of one cannabinoid is about 5 mg/dose and the total weight of a second cannabinoid is about 5 mg/dose.

17. The composition of any one of claims 1-14, wherein at least two cannabinoids are in a 10:1 proportion by weight.

18. The composition of any one of claims 1-14, wherein at least two cannabinoids are in a 20:1 proportion by weight.

19. The composition of any one of claims 1-14, wherein the total weight of one cannabinoid is between 0.01 mg/dose and 0.5 mg/dose and the total weight of a second cannabinoid is between 9 mg/dose and 10 mg/dose.

20. The composition of any one of claims 1-14, wherein the total amount of one or more cannabinoids is between about 0.1 grams and about 10 grams.

21. The composition of any one of claims 1-14, wherein at least one cannabinoid is THC and is present in an amount ranging from about 0.1 mg to about 10 mg.

22. The composition of any one of claims 1-14, wherein at least one cannabinoid is CBD and is present in an amount ranging from about 0.1 mg to about 10 mg.

23. The composition of any one of claims 1-14, wherein the total amount of one or more cannabinoids or cannabinoid extracts are present in an amount of about 1% to 2% by weight of the composition.

24. The composition of any one of claims 1-14, wherein the total amount of one or more cannabinoids or cannabinoid extracts are present in an amount of about 1.5% by weight of the composition.

25. The composition of any of claims 1-14, wherein said composition is a pharmaceutical composition or formulation.

26. The pharmaceutical composition of claim 25, wherein said composition is formulated for penetrating the mucosal barrier.

27. The pharmaceutical composition of claim 25, wherein said composition is formulated for rapid disintegration upon administration.

28. The pharmaceutical composition of any one of claims 26-27, wherein said composition is formulated in a dosage form.

29. The dosage form of claim 28, wherein said unit dose is selected from an oral dosage form, a sublingual dosage form, or a buccal dosage form.

30. The dosage form of any one of claims 28-29, wherein said composition is a liquid.

31. The dosage form of any one of claims 28-30, wherein said dosage form is a tincture.

32. The dosage form of claim 31, wherein at least two cannabinoids are in a 20:1 proportion by weight.

33. The dosage form of claim 31, wherein the first cannabinoid weighs between about 10 mg/ml and 10.4 mg/ml and the second cannabinoid weighs between about 0.47 mg/ml and 0.6 mg/ml.

34. The dosage form of claim 31, wherein the first cannabinoid weighs about 0.56 mg/ml and the second cannabinoid weighs about 12.6 mg/ml.

35. The dosage form of claim 31, wherein at least two cannabinoids are in a 10:1 proportion by weight.

36. The dosage form of claim 31, wherein at least two cannabinoids are in a 1:1 proportion by weight.

37. The dosage form of claim 31, wherein the first cannabinoid weighs between about 4.9 mg/ml and 5.5 mg/ml and the second cannabinoid weighs between about 4.9 mg/ml and 5.5 mg/ml.

38. The dosage form of any one of claims 32-37, wherein the first cannabinoid is THC and the second cannabinoid is CBD.

39. The dosage form of claim 31, wherein said tincture is contained in a bottle for periodic administration.

40. The dosage form of claim 39, wherein the first cannabinoid weighs between about 70 mg and 72.8 mg and the second cannabinoid weighs between about 3.29 mg and 4.2 mg.

41. The dosage form of claim 39, wherein the first cannabinoid weighs between about 34.3 mg and 38.5 mg and the second cannabinoid weighs between about 34.3 mg and 38.5 mg.

42. The dosage form of claim 39, wherein the first cannabinoid weighs about 3.92 mg and the second cannabinoid weighs about 88.2 mg.

43. The dosage form of any one of claims 40-42, wherein the first cannabinoid is THC and the second cannabinoid is CBD.

44. The dosage form of claim 31, wherein said dosage form contains from about 0.1 to about 10 mg of total cannabinoids or cannabinoid extracts.

45. A method for treating a disease comprising administering a daily therapeutically effective amount of the pharmaceutical composition according to any one of claims 25-28 or the dosage form according to any one of claims 29-44.

46. The method of claim 45, wherein the disease is selected from pain associated with cancer, neuropathic pain and HIV-associated sensory neuropathy, side effects of chemotherapy including nausea and pain, symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia.

47. A method of sublingually or buccally administering a therapeutic agent to a patient comprising the steps of:
a) preparing a pharmaceutical composition as defined by any one of claims 25-28, wherein the pharmaceutical composition is a liquid,
b) administering the pharmaceutical composition to the underside of the tongue of the patient.
